(19) United States
(12) Patent Application Publication
(10) Pub. No.: US 2003/0156976 A1
(43) Pub. Date: Aug. 21, 2003

(54) Cleansing blocks

(75) Inventors: Brian Wilson, Diss (GB); Steve Balls, North Pickenham (GB); Darren Smith, Thetford (GB); Peter Virica, Newscastle-upon-Tyne (GB); Ian Sidgwick, Norwich (GB)

Correspondence Address: BLANK ROME COMISKY & MCCAULEY, LLP, 900 17TH STREET, N.W., SUITE 1000, WASHINGTON, DC, 20006, US

(73) Assignee: JEYES GROUP LIMITED, Thetford (GB)

(21) Appl. No.: 10/361,847

(22) Filed: Feb. 11, 2003

(30) Foreign Application Priority Data
Feb. 11, 2002 (WO) PCT/GB02/00599
Apr. 17, 2002 (GB) 0208794.8

(51) Int. Cl. A01N 1/00; A61L 2/00

(52) U.S. Cl. 422/28; 422/37; 422/261

(57) ABSTRACT

A cleansing block for treating water comprises from 40% to 80% by weight of a halogen release agent, from 20 to 55% by weight of a surfactant component, and from 1 to 10% by weight of a hydrophobe.

### INTRODUCTION AND BACKGROUND

This invention relates to cleansing blocks for cleansing or disinfecting water, and in particular for cleansing blocks used in lavatories. Lavatory cleansing blocks incorporating a bleach component are well known. Typically they are immersed in the water in the toilet cistern, or they are suspended in a chamber under the rim of the toilet bowl. The different types of use require different formulations and much work has been done in developing formulations which will provide an adequate life time in use (typically three to four weeks) but also a sufficiently long shelf life as the bleach component may decompose and/or cause discolouration of the block.

In some uses the block may incorporate additional components such as a surfactant which provides foam—as visible evidence to the user that a cleaning action is underway as well as providing a detergent action; a perfume to provide a pleasant smell; and a dye to provide a visible indicator that the block is active as well as distinct in appearance. The block may also include various production aids such as plasticizers to assist in extrusion of the block, solubility control agents to prolong the lifetime of the block, and fillers such as salts.

When formulating the block it is also necessary to take into account the production process, as compaction or extrusion of high bleach content blocks can be hazardous.

We have developed a lavatory cleansing device in which a solid block of material is held in a relatively enclosed chamber at the toilet rim, a small quantity of water from the toilet flush is directed into the chamber having the block and remains in contact with the block for a period of a few minutes while it drains slowly out of the chamber, into the toilet bowl. The block is therefore subject to somewhat hybrid conditions in not being fully immersed in water for a prolonged period of time, like the in-tank block, and not being exposed only briefly to the passing flush water. To meet the requirements for this new block environment we have developed a novel formulation for the solid lavatory cleansing block.

### SUMMARY OF THE INVENTION

The main function of the block is to provide a cleansing action using a bleach component and so the block comprises from 45 to 80% by weight of a bleaching agent. Very high bleach content blocks are known per se, but we have found that they tend to be too soluble—and so have a short life time, and to swell in use—making them unsuitable for use in an enclosed housing. The bleaching agent is preferably a halogen release agent. To control the solubility of the block we incorporate a surfactant component which makes up substantially most of the remainder of the active block ingredients, and in particular from about 20% to 55% by weight of the block content. The surfactant component may be a mixture of surfactants and preferably includes an anionic surfactant.

We further provide a small amount of a hydrophobe, preferably less than about 10% by weight, and more preferably less than about 6% by weight of the block. The hydrophobe can provide two functions, as a processing aid and to prolong the in use life of the block, slowing dissolution and preventing swelling.

Preferably other components in the block make up less than about 150% by weight of the block composition.

The halogen release agent is preferably a chlorine release agent and sodium dichloroisocyanurate is particularly preferred but a chlorohydantoin such as Dantochlor (T.M.) may also be used. EP-A-206 725 and EP-A-817 832 mention several high halogen content agents such as dichlorodimethyihydantoin (DCDMH) and bromochlorodimethylhydantoin (BCDMH), but used in a low amount. Suitable surface active agents for use in the composition of the invention include but are not limited to anionic surface active agents, especially alkali metal, typically sodium alkyl sulphates (e.g. lauryl sulphate), primary and secondary alkane sulphonates or sarcosinates or mixtures thereof. Preferably these are in substantially non-oxidizable form, and more preferably are in substantially anhydrous form. In this context, “substantially anhydrous” means that the water content as expressed as the total of free and banded water in the surfactant is less than 2% by weight of the surfactant sample, more preferably less than 1% by weight.

It is particularly preferred to use a pliable surfactant which will further said processing such as in extrusion, and a secondary alkane surfactant is an example of a preferred surfactant for this.

The solubility control agent when provided should also preferably be substantially non-oxidizable. It is selected from one or more such materials. Provided that this criterion is met, such agents may be virtually wholly insoluble in water or if, as discussed below, a nonionic surface active agent, have a low HLB, e.g. 5 or less. Such agents being substantially non-oxidizable are substantially resistant to attack by the halogen release component, both in the composition and in aqueous solutions produced by dissolution of the composition in use. It is a matter of simple experiment to determine whether any candidate is so resistant. Generally, the solubility control agent should be a saturated organic material or a highly chlorinated organic material. Examples of solubility agents which may be employed include but are not limited to polyethylene waxes; saturated tertiary alcohols; paradichlorobenzene; and difficulty hydrolysable esters.

Solubility control agents may also be chosen from non-oxidizable organic liquids. A wide variety of these may be employed, such as mineral oils, liquid hydrocarbons (e.g. liquid alkanes), chlorinated hydrocarbons, silicone oils, liquid ketones (e.g. 2-decanone), liquid tertiary alcohols (e.g. 2-methyl-bexan-2-ol), and liquid esters e.g. simple esters such as methyl decanoate, and more complex esters such as glycerol, propylene glycol, triethylene glycol esters of C₈-C₁₀ fatty acids and/or succinic acid. Examples of such complex esters are those sold under the trade name Miglyol 812, Miglyol 829, Miglyol 840, Plasthall 4141, Crodamol GTCC, Crodamol PC and Radia 7108.

Particularly preferred examples of such solubility control agents include solid esters; waxes; saturated fatty acid esters; mineral oils; and polysiloxanes. It has been found that the use of a mixture of non-oxidizable solubility control agents may often prove useful. Thus a mixture of an essentially hydrocarbon material, such as paraffin wax and/or a mineral oil, with an ester of a fatty acid such as isononyl stearate or triethylene glycol caprate/caprylate, may be employed. Further, it is sometimes useful to employ, in admixture, a combination of both a solid and a liquid hydrocarbon material. Other substantially non-oxidizable solubility control agents include glycolurils and inorganic compounds such as aluminium hydroxide.

As will be appreciated, any other ingredient present in the compositions of the invention should preferably be substantially resistant to attack by the halogen release agent. Most perfumes which are commonly employed in lavatory cleansing blocks are also subject to attack by halogen-release agent although some odiferous materials may be adequately resistant (and additionally serve as solubility control agents). Adequately resistant examples of these include substituted quinolines, cedryl methyl ether and cineole. Many of these perfumes are suitable also to fulfil the role of the solubility control agent as well as to function as processing aids, e.g. in an extrusion process. Particular examples of perfumes include Citrus 4022 ex IFF, Pine 2363 ex IFF, Fantasy 2366 ex IFF, Artic Fresh 2662 ex IFF, and Oriental Fruits F540096 ex Quest, all these names being Trade Marks.

Fillers, typically mineral fillers, may be present in the block and are suitably present in amounts of from 0 to 10% by weight, preferably 0.5% to 6% by weight, more preferably 1 to 3% by weight. The filler is often required to increase the density, life or integrity of the block and suitable examples are salts such as calcium sulphate and sodium sulphate, as well as clays such as laponite.

Lavatory cleansing blocks commonly contain a germicide or preservative but this is not generally necessary in the case of the blocks of the invention since they already contain a powerful germicide(s), namely the halogen-release agent.

Some insoluble pigments are resistant to halogen release agents and may be incorporated in the blocks of the invention to impart a coloration.

Suitable examples include inorganic pigments of mixed—phase types, consisting exclusively of metal oxides such as Co/Al oxide (blue) and Co/Ti/Ni/Zn oxides (Green). These can be conveniently incorporated in the blocks of the invention by dispersing in the powder mixture.

Solid blocks may suitably be formed by a compression process, especially an extrusion or tabletting process. For extrusion, this comprises the steps of forming a mixture of the components of the composition, extruding this mixture into rod or bar form and then cutting the extruded rod or bar into appropriately sized pieces or blocks.

Alternatively, blocks may be prepared by a melting and casting process, e.g. one in which fusible components of the block are fuse melted, the non-fusible components adhere to the melt, and the resultant mixture cast into moulds.

When an extrusion process is employed the mixture to be extruded should preferably contain up to about 5% by weight liquid component or a solid component which is liquefied under extrusion conditions to act as a processing aid. Suitable such liquids include hydrocarbons (e.g. liquid alkanes), polysiloxanes, chlorinated hydrocarbons, silicone oils, liquid ketones (e.g. 2-decanone), liquid tertiary alcohols (e.g. 2-methyl-hexan-2-ol), and liquid esters e.g. simple esters such as methyl decanoate, and more complex esters such as glycerol, propylene glycol, triethylene glycol esters of C₈-C₁₀ fatty acids and/or succinic acid. Examples of such complex esters are those sold under the trade names Miglyol 812, Miglyol 829, Miglyol 840, Plasthall 4141, Crodamol GTCC, Crodamol PC and Radia 7108. It will thus be appreciated that these liquid components can also serve as solubility control agents as well as processing aids and a component having this dual role is particularly preferred. Suitable liquefiable solid components include solid esters such as cetyl palmitate and these also can serve as solubility control agents.

### DESCRIPTION OF THE PREFERRED EMBODIMENTS

In order that the invention may be well understood the following examples are given by way of illustration only. All amounts are expressed as percentages by weight unless explicitly recited to the contrary. Typical block weights are about 75 gm.

TABLE 1

| | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|
| Dantochlor | | | | | | | | | | | | | | | | 65 |
| Ufaryl DL90 | | 20 | 20 | | | | | 15 | 20 | | | | | | | |
| Ufaryl DL80P | 20 | | | 20 | 20 | 17.5 | 20 | | | 17.5 | 17.5 | 15 | 15 | 15 | 12 | 15 |
| Hostapur SAS 93G | 20 | 20 | 20 | 20 | 20 | 17.5 | 20 | 15 | 20 | 17.5 | 17.5 | 15 | 15 | 16.5 | 12.5 | 15 |
| S.D.I.C. | 45 | 45 | 50 | 50 | 50 | 55 | 45 | 45 | 40 | 55 | 60 | 65 | 65 | 65 | 70 | |
| Calcium Sulphate s.f.w. | 10.3 | 5.5 | | | 5.5 | 5.5 | 10.3 | 21.3 | 15.3 | | | 1.5 | | | 1.5 | 1.5 |
| Plasthall 4141 | 1 | 1 | 1 | 1 | 1 | 1 | 1 | 1.0 | 1 | 1 | 1 | 1 | 1 | 1 | 1.5 | 1 |
| Mineral Oil | 3.5 | 3.5 | 3 | 3.5 | 3.5 | 3.5 | 3.5 | 2.5 | 3.5 | 3 | 3 | 2.5 | 2.5 | 2.5 | 2.5 | 2.5 |
| Tytanpol R-002 | 0.2 | — | | | | | 0.2 | 0.2 | 0.2 | | | | | | | |
| Sodium Sulphate | | 5 | 6 | 5.5 | | | | | | 6 | 1 | | 1.5 | | | |

| Raw Material | Chemical Name | CAS Number |
|---|---|---|
| Dantochlor | 1,3 dichloro 5,5 dimethylhydantoin (81%) | (118-52-5) |
| | 1,3 dichloro 5 ethyl 5 methyl imadazolydine 2,4 dione (18%) [proportions may vary] | (89415-87-2) |
| Unger Ufaryl DL90 | Sodium Alkylaryl Sulphonate (Sodium Dodecylbenzene Sulfonate)(90% active) | (25155-30-0) |
| Hostapur SAS 93G | Secondary alkane sulphonate, sodium salt (92% active) | 85711-69-9 |
| Ufaryl DL80P | Sodium Alkylaryl Sulphonate (Sodium Dodecylbenzene Sulfonate)(80% active) | (25155-30-0) |
| Sodium Sulphate | Sodium Sulphate | 7757-82-6 |
| Mineral Oil | White mineral oil, or Paraffin Oil | |
| Plasthall 4141 | Triethylene Glycol Caprate Caprylate | 68130-48-3 |
| Calcium Sulphate s.f.w. | Gypsum, or Calcium Sulphate dihydrate | |
| S.D.I.C. | Sodium Dichloroisocyanurate, dihydrate | 51580-86-0 |

Blocks 10 to 15 were held in a dispenser of the type described in PCT/GB0210059 as will be described more fully hereinafter. During each flush, about 5 cm³ of water enters the tablet chamber and remains for a while as it drains slowly from the chamber. The tablets lasted for about 500 flushes, equivalent to several weeks use.

The invention will be further described by way of example only, with reference to the accompanying drawings, in which:

FIG. 1 is a front perspective view of a lavatory cleansing device forming a first embodiment of the invention;

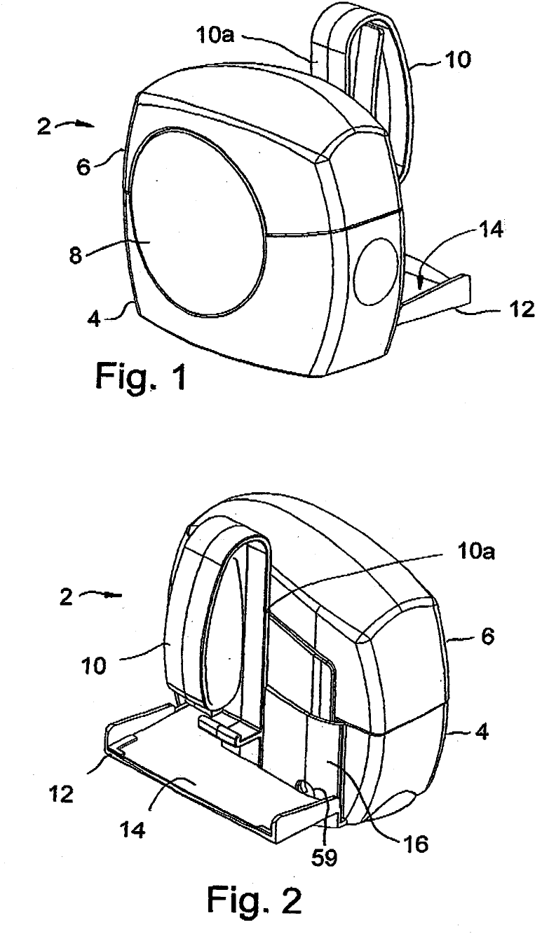

FIG. 2 is a rear perspective view of the device of FIG. 1;

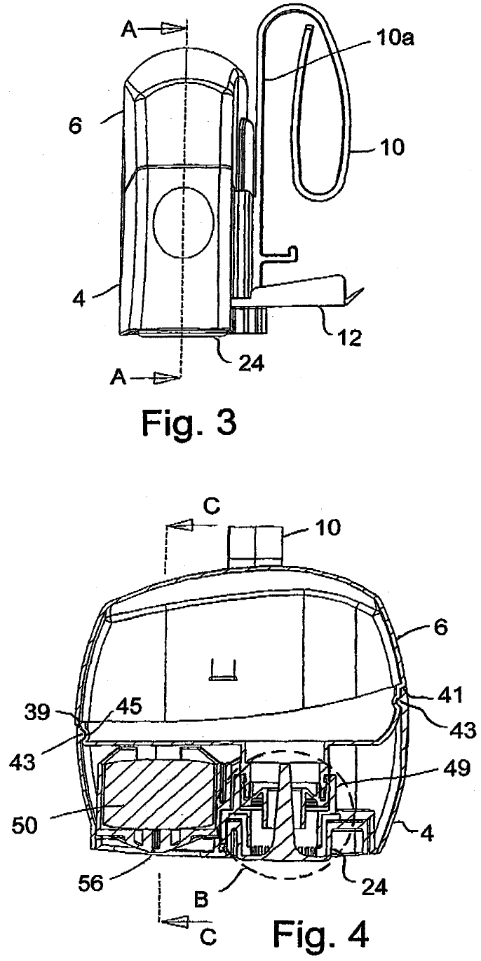

FIG. 3 is a side view of the device of FIG. 1;

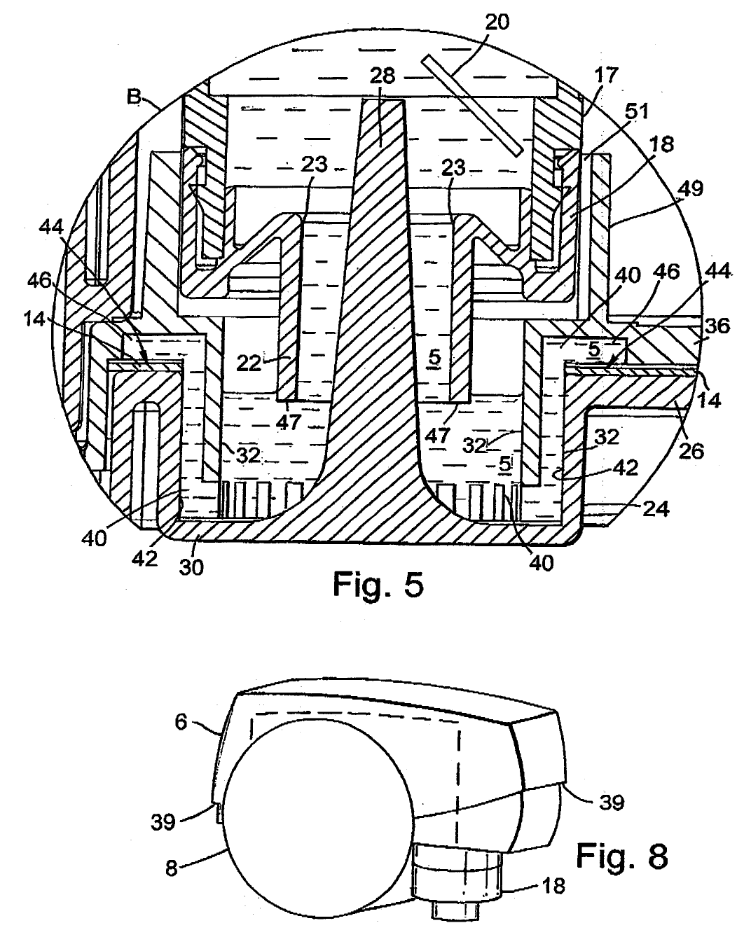

FIG. 4 is a cross-section along the line A-A of FIG. 3;

FIG. 5 is an enlarged detail of FIG. 5, on circle B;

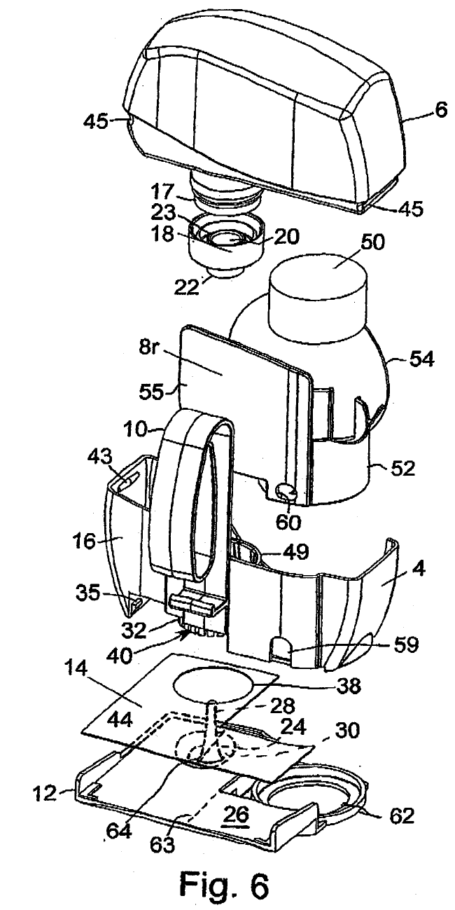

FIG. 6 is an exploded view of the device of FIG. 1;

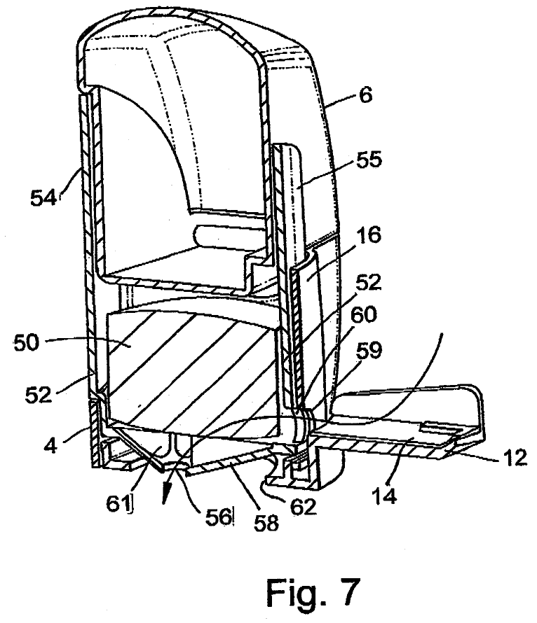

FIG. 7 is a perspective view on line C-C of FIG. 4;

FIG. 8 is a perspective view of a refill for the device of FIG. 1;

FIG. 9 is a side view of an embodiment of a modified tablet holder for the embodiment of FIGS. 1 to 8;

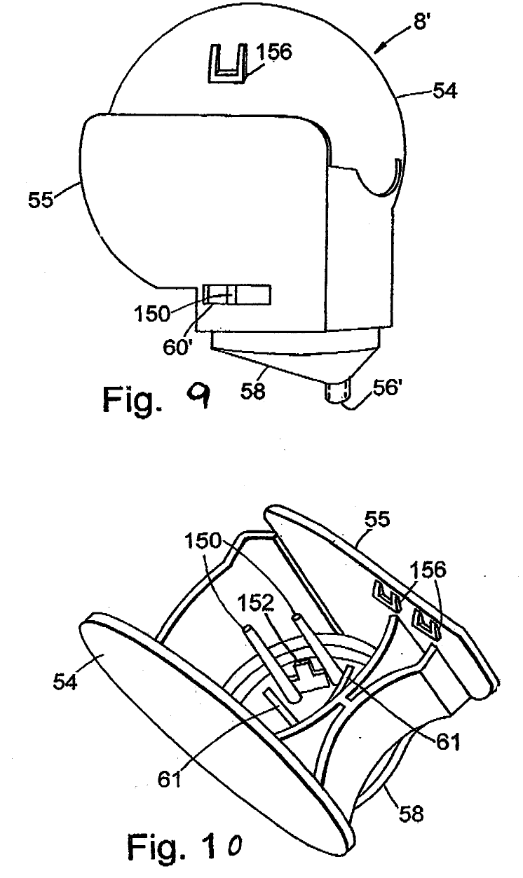

FIG. 10 is a perspective view from above of the holder of FIG. 9, and

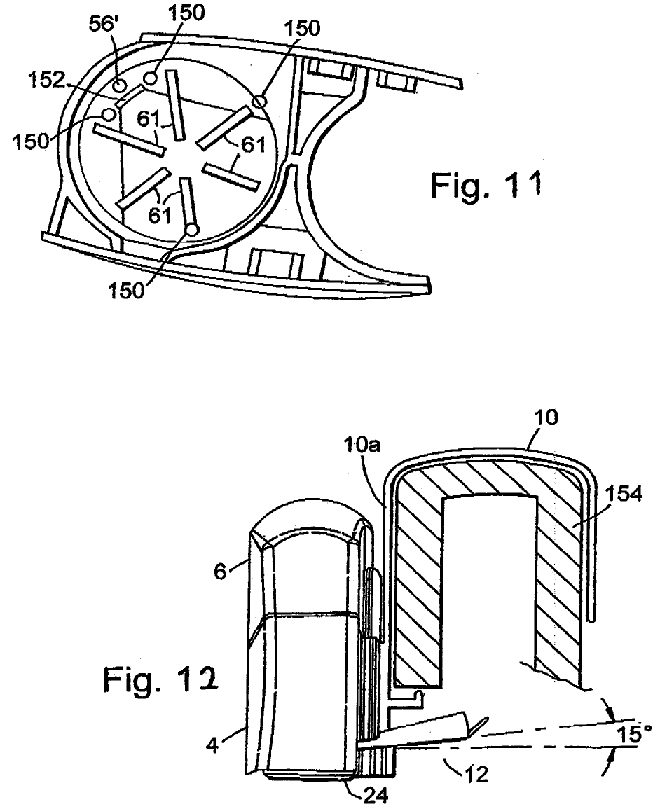

FIG. 11 is a plan view of the tablet holder of FIG. 9, and

FIG. 12 is a side view corresponding to FIG. 3 and showing a sloping delivery plate and the device mounted on a rim of a toilet bowl.

In the embodiment of FIGS. 1 to 8, a lavatory cleansing device 2 comprises a main support member 4 which carries a container 6 containing a liquid formulation 5 (see FIG. 5) and a chamber 8 which houses a solid formulation. Container 6 and chamber 8 are clipped together and are removable from the mains support member 4. The combination of container 6 and chamber 8 form a refill, as seen in FIG. 8.

Support member 4 has a folded strap 10 on its rear wall 16. Strap 10 is unfolded and hooked over the rim of a toilet bowl, as is well known in the art. Strap portion 10a will bear against the inner wall of the toilet rim (see FIG. 16).

As more clearly seen in FIGS. 2 and 6, a delivery plate 12, having a textile layer 14 on its upper surface, is attached to the underside of the support member 4 and extends to the rear of the rear wall 16. In use, plate 12 will extend underneath the rim of the toilet bowl so that flush water will splash over the textile layer 14.

Plate 12 may be configured to abut the side wall of the toilet bowl to ensure collection of sufficient water in the toilet bowls, particularly US style bowls, in which the flush water is directed to flow down the wall rather than cascade turbulently from the rim.

As will be described more fully hereinafter, the lavatory cleansing device 2 incorporates, in effect, two delivery systems, a first for delivering a liquid product and a second for delivering components from a solid product, whilst keeping the products separate and allowing for a single refill unit.

The liquid delivery system will be described first. Container 6 is typically moulded of clear or semi-transparent PET or PVC. High density polyethylene or polypropylene may be used if the container contains, or is contacted by a bleach component, for example. Container 6 has an outlet opening 17 closed by a cap 18 which has a fangible seal 20 and a tubular collar 22 extending down below the seal 20. Prior to fracture, seal 20 extends across the inner end 23 of collar 22.

Delivery plate 12 moulded of polypropylene has a cup 24 which depends downwardly from a main supporting surface 26. A spigot 28 projects up from the bottom wall 30 of the cup 24.

The layer 14 of the textile material lays on the supporting surface 26. Textile material layer 14 may be glued or welded to surface 26, for example by ultrasonic welding or held flat by spikes projecting up from surface 26, for example.

Textile layer 14 has an aperture 38 which coincides with the mouth of cup 24. Textile layer 14 need not fully surround cup 24. The requirement is to deliver liquid from the cup to the exposed area of the plate 26 and textile layer 14.

Main support member 4 has a capillary member 32 integrally formed on a bottom wall 36 of support member 4. Capillary member 32 extends through aperture 38 of textile layer 14, into the cup 24. Capillary member 32 is cylindrical, and has capillary channels 40 formed in its outer wall, facing the inner wall 42 of cup 24. Capillary channels 40 extend up to the underside of bottom wall 36, and along a thickened region of wall 36, forming an L-shape which will overlap the textile layer 14. Capillary channels 40 are positioned to deliver liquid onto the textile layer 14.

Delivery plate 12, with textile layer 14 in position, is clipped to support member 4, for example by nibs 35 integrally formed on the walls of support member 4, which clip under delivery plate 12.

In use, the container 6 is pushed into support member 4, spigot 28 displacing the frangible seal 20 of cap 18. A ledge 39 formed in the wall of container 6 rests on the upper edge 41 of support 4. Ribs 43 on the inside surface of support 4 engages in recesses 45 in the container wall. Liquid 5 will flow from the container 6 into cup 24, through collar 22. Liquid from cup 24 is ducted up onto the upper surface 44 of textile layer 14 by the capillary channels 40. Capillary channels 40 co-operate with the inner wall 42 of cup 24 to duct the liquid upwards, until it passes into the upper leg 46 of capillary channels 40, which extend over the fabric upper surface 44.

The liquid is then ducted away from the capillary channels 40 by the texture of the textile, to spread slowly over the surface 44. It is preferred that there is no substantial absorption into the textile layer, thus a hydrophobic treatment may be preferred. One example of a textile layer is knitted on a Karl Meyer Raschel Jacquard=RMJG 5 FNE Machine, Warp (Pillar) 150/36 Semi Dull Polyester (Flat) Back Bar (Top Creel) 1/167 Textured Polyester, Front Bar (Bottom Creel) 2/167 Textured polyester. The presently preferred material is a Jacquard knitted polyester of open weave, having

| Motions | A | B |
|---|---|---|
| Front Bar | Cut | Cut |
| Back Bar | Cut | Miss |

With a quality of 25.4 C.P.I.

Another fabric type is

| Motions | A | B |
|---|---|---|
| Front Bar | Miss | Cut |
| Back Bar | Cut | Miss |

With a quality of 25.4 C.P.I.

This arrangement is particularly suited for use with viscous liquids. For less viscous liquids, the textile layer 14 may be dispensed with, and the upper surface 26 of plate 12 may be textured, for example with radial grooves, to duct liquid over the surface. Initially, the liquid from container 6 will fill the cup 24 only to just above the lower edge 47 of the collar 22. As the liquid is ducted out of the cup 24 by the capillary channels 40, the level of liquid in cup 24 falls below the edge 46, allowing air to enter container 6, and so allowing more liquid to flow from the container into cup 24 until edge 46 is covered again.

Container cap 18 fits in a cylinder 49 extending up from the wall 36. Grooves 51 are provided on the inside of the wall of cylinder 49 to allow air to enter cup 24. Wall 36 sits just clear of textile layer 14 to allow liquid 5 to flow out over the surface of the textile. It has been found that the spacing of the wall 36 from the supporting surface 26, and hence the degree of compression, if any, of the textile 14 affects the rate of delivery of the product from the well or cup 24. Hence some trial is required to optimise the spacing to suit the particular textile, liquid viscosity., etc.

When the device is installed in a toilet bowl, flush water will flow over upper surface 44 of textile layer 14, washing the liquid from the textile surface.

Preferably, the liquid viscosity, capillary channel size, fabric surface, etc. are tailored to replenish the surface 44 with a fresh dosage of liquid within 30 seconds to 10 minutes after the toilet bowl has been flushed.

The above liquid delivery arrangement is described in more detail in co-pending applications Nos. WO 01/32995 and GB 0026832.6, the contents of which are incorporated herein by reference.

The other component of the cleaning device 2 is a solid tablet 50 of bleaching agent as described with reference to example 1 to 18 above.

Tablet 50 is held in chamber 8 which fits in support member 4, under container 6. Chamber 8 is moulded of polypropylene and forms a cylindrical cup 52 in which tablet 14 sits. Front and rear walls 54, 55 extend up from the cup 52 to embrace the container 6.

Chamber 8 is configured to receive a small amount of flush water which percolates around tablet 50 to dissolve the bleach component and then drips into the toilet bowl. It is desirable to separate the bleach component from the liquid component of container 6 until they mix in the toilet bowl. Thus, flush water exits chamber 8 at an aperture 56 in the bottom wall 58 of cup 52 as seen most clearly in FIG. 7.

Referring to FIG. 7, an aperture 59 is formed in the rear wall 16 of support member 4, level with the textile layer 14 on the delivery plate 12. A corresponding aperture 60 in cylindrical cup 52 is aligned with aperture 59. Flush water flowing onto the textile layer 14 will splash through apertures 59, 60 and on to tablet 50. The water will then flow down and out through aperture 56. Tablet 50 is supported on ribs 61, to support the tablet over the conical bottom wall 58 of cup 52. Bottom wall 58 is aligned with an aperture 62 in delivery plate 12.

The textile layer 14 is fixed to delivery plate 12, which is then snapped in place on the underside of the supporting member 4. The chamber 8 with a bleach tablet 50 in place is snapped onto the container 6, which is filled with liquid surfactant mixture and closed by cap 18. At the point of use, the consumer pushes the container/chamber unit into the supporting member 4. Spigot 28 displaces seal 20, so that liquid fills cup 24 and is ducted onto the surface 44 of textile layer 14 as described above. The unit is suspended at the rim of a toilet bowl, plate 12 extending into the flow of flush water.

When the toilet is flushed, the flush water rapidly washes liquid 5 from the surface of the textile layer 14. Water will also pass through apertures 59, 60 to flow over bleach tablet 50 and wash bleach components out through aperture 56. Although the flush water may carry some of the surfactant formulation 5 over the bleach tablet 50, this will be washed out through aperture 56 and so there will not be prolonged contact in cup 52. In any event, any discoloration at tablet 50, for example, would not be visible to the user.

Plate 12 may be partitioned at line 63 of FIG. 6, or the fabric layer stopped short of the apertures 59 for example at line 64 of FIG. 6.

As indicated above, it is desirable to provide a liquid formulation in container 6 comprising surfactant, perfume, dye and optionally a disinfecting agent. EP-A-775 741 describes such formulations.

| | % by Weight |
|---|---|
| Anionic Surfactant | 26.5 |
| Nonionic Surfactant | 5.0 |
| Solvents | 10.0 |
| Antioxidant | 0.004 |
| Perfume | 10.0 |
| Dye | 0.0024 |
| Preservative | 0.1 |
| Natrosol Cellulose Thickener | 0.4* |
| Water | Balance |

The amount of natrosol is varied to achieve the desired viscosity, which typically is in the range 350 to 5000 centipoise, preferably 2000 to 4000, measured in a Broomfield LV viscosimeter, 20° C., spindle 2, speed 6.

FIGS. 9 to 11 show a modified tablet holder for the embodiment of FIGS. 1 to 8. This tablet holder is arranged to hold the flush water, which enters the holder, in contact with the tablet for a short period of time prior to releasing it into the toilet bowl. By having a noticeable residence period in the tablet holder, the flush water will take components of the bleach block (or other block type in the holder) into solution in a more controlled manner. Also, by delaying the drainage of the formed solution into the toilet bowl, the constituents are less likely to be carried out of the bowl with the flushing water.

To achieve a residence time in the tablet holder we provide an outlet aperture of small size, arranged to allow water to drain from the tablet holder. It is desirable to arrange for the solution to drip into the toilet bowl while there is still some turbulence in the water in the bowl but after the flow of water out of the bowl has ended. To ensure that a sufficient quantity of water enters the tablet holder, the plate 12 is sloped downwards towards the entry aperture 59, 60, preferably at an angle of about 10 to 20 degrees to the horizontal, and preferably about 15 degrees, as seen in FIG. 16. The horizontal may be taken as the perpendicular to the main strap portion 10a (FIGS. 1 and 2). It will be appreciated that the actual angle of the plate 12 to the horizontal will depend on the orientation adapted by the device when installed by the user on the toilet bowl rim 154 and this itself may also vary between bowl designs.

FIG. 9 shows a perspective view of the tablet holder 8′ from the rear side (i.e. the side of the delivery plate 12 when installed) cf. the view in FIG. 6. Inlet aperture 60′ aligns with the upper surface of the plate 12 or fabric layer 24 and aperture 59 in the rear wall of the holder 4 (see FIG. 6). Aperture 60′ is dimensioned to allow the tablet holder 8′ to fill to about the level of the aperture 60′ during a flushing cycle. Water filling the tablet holder 8′ eventually drains from the tablet holder through outlet aperture 56′ in the bottom wall 58. In this embodiment, the outlet aperture is about 1.3 mm across, typically starting to drain a few seconds after the flush has stopped. The major portion of the water will flow out in the first minute, typically 95% in 40 seconds, and the chamber will continue to drain for several minutes, preferably ten minutes or more after the flush. The final drops of water from the chamber will have a high bleach concentration and so add significantly to the bleach in the toilet bowl. Outlet aperture 56′ will be smaller than inlet aperture 60′. The quantity of water in tablet holder 8′ will depend on the water flow (smooth or turbulent) and amount of tablet left, but a quantity in the region of 5 to 20 cm³ is preferred.

As with the embodiment of FIGS. 1 to 8, bottom wall 58 is dished, sloping down to the outlet aperture 56′, and a tablet 8, is supported on radial ribs 61. A plurality of pins extend up from the bottom wall 58 and the tablet is located between the pins 150. This ensures that the tablet is held clear of the inlet and outlet apertures 60′, 56, to avoid blocking them, and water can access the side and underneath of the tablet. Also, a shield plate 52 further protects the outlet aperture 56′ to prevent tablet particles blocking the aperture.

Nibs 156 engage in recesses in the sidewalls of container 6 to clip the chamber 8 to the container, to form a refill unit.

We formulate a block or tablet 8 which will dissolve slowly in the flush water and which will not swell or break up to a significant extent, otherwise there is a risk of blocking the inlet or outlet apertures 56, 60, 56′, 60′.

Tablets of formulations 10 to 15 above showed good performance in the case of FIGS. 9 to 11 deliver a dose of bleach component into the toilet bowl for up to 500 flushes (about 4 weeks use). Compositions 12 and 13 gave particularly good performance.

What we claim is:

1. A cleansing block for water, comprising from 40% to 80% by weight of a halogen release agent, from 20 to 55% by weight of a surfactant component, and from 1 to 10% by weight of a hydrophobe.

2. A block as claimed in claim 1, having from 1 to 6% by weight of the hydrophobe component.

3. A block as claimed in claim 2, having from 4 to 6% by weight of the hydrophobe component.

4. A block as claimed in claim 2, wherein the hydrophobe includes an oil.

5. A block as claimed in claim 4, wherein the oil is in an amount of from 2 to 4% by weight of the block.

6. A block as claimed in claim 4, having from 2.5 to 3.5% by weight of the oil.

7. A block as claimed in claim 1, wherein the surfactant includes a secondary alkane sulphonate.

8. A block as claimed in claim 7, wherein the secondary alkane sulphonate is present in an amount of from 10 to 30% by weight.

9. A block as claimed in claim 7, wherein the secondary alkane sulphonate is present in an amount of from 12.5 to 20% by weight.

10. A block as claimed in claim 9, having about 15% by weight of the secondary alkane sulphonate surfactant.

11. A block as claimed in any one of claims 1, having from 10 to 30% by weight of a second surfactant.

12. A block as claimed in claim 11, having from 12.5 to 20% by weight of the second surfactant.

13. A block as claimed in claim 12, having about 15% by weight of the second surfactant.

14. A block as claimed in claim 11, wherein the second surfactant is a secondary alkylaryl sulphonate.

15. A block as claimed in claim 1, wherein the halogen release agent is a chlorine release agent.

16. A block as claimed in claim 15, wherein the halogen release agent is an isocyanurate.

17. A block as claimed in claim 15, having from 60 to 70% of the halogen release agent.

18. A block as claimed in claim 17, having about 65% of the halogen release agent.

19. In combination, a cleansing block according to claim 1 and a dispenser for holding the block and capturing water flow to dissolve materials of the block.

20. The combination of claim 19, including a strap for holding the dispenser on the rim of a toilet bowl.

21. A method of treating water, comprising contacting a block of claim 1 with a portion of water to dissolve part of the block and form a solution, and draining the solution into the water to be treated.
